(19) United States
(12) Patent Application Publication
(10) Pub. No.: US 2010/0023086 A1
(43) Pub. Date: Jan. 28, 2010

(54) IMPLANTABLE PULSE GENERATOR EMI FILTERED FEEDTHRU USING DISCRETE CAPACITORS

(75) Inventor: Wisit Lim, Palmdale, CA (US)

Correspondence Address: PACESETTER, INC., 15900 VALLEY VIEW COURT, SYLMAR, CA, 91392-9221, US

(73) Assignee: PACESETTER, INC., Sylmar, CA (US)

(21) Appl. No.: 12/179,503

(22) Filed: Jul. 24, 2008

(51) Int. Cl. A61N 1/362; H01G 4/35

(52) U.S. Cl. 607/36; 607/72; 361/302

(57) ABSTRACT

Disclosed herein is an EMI filtered feedthru for an implantable pulse generator. The EMI filtered feedthru may include a filter assembly, which has a chip capacitor and a body. The body may include a cavity in which the chip capacitor resides.

### FIELD OF THE INVENTION

The present invention relates to medical apparatus and methods. More specifically, the present invention relates to feedthrus for implantable pulse generators and methods of manufacturing such feedthrus.

### BACKGROUND OF THE INVENTION

Implantable pulse generators, such as pacemakers, defibrillators or implantable cardioverter defibrillators (“ICD”), are used to provide electrotherapy to cardiac tissue via implantable medical leads. An implantable pulse generator feedthru is used for an electrical pathway extending between the electrically conductive lead securing components of a header of the pulse generator and the electrical components, such as an output flex, hybrid, etc., hermetically sealed in the housing or can of the pulse generator.

Feedthrus are mounted in the wall of the housing or can and include feedthru wires extending through the feedthrus. Feedthrus provide insulated passageways for feedthru wires, such as platinum iridium (Pt/Ir) wires, through the wall of the can. The header ends of the feedthru wires are electrically connected to connector blocks that mechanically and electrically couple with connector ends of implantable medical leads, and the can ends of the feedthru wires are electrically connected to the electrical components housed in the can of the pulse generator.

Feedthrus may include a filter element to filter out unwanted signals, such as electromagnetic interference (“EMI”). Current feedthrus employ discoidal filter assemblies as the EMI filter element. Discoidal filter assemblies are disadvantageous due to high associated material and manufacturing costs.

There is a need in the art for a feedthru that has reduced material and manufacturing costs. Also, there is a need in the art for a method of manufacturing such a feedthru.

### BRIEF SUMMARY OF THE INVENTION

Disclosed herein is an EMI filtered feedthru for an implantable pulse generator. In one embodiment, the EMI filtered feedthru includes a filter assembly, which has a chip capacitor and a body. The body includes a cavity in which the chip capacitor resides.

Disclosed herein is an implantable pulse generator. In one embodiment, the pulse generator includes an EMI filtered feedthru. The EMI filtered feedthru may include an EMI filter assembly having a chip capacitor and a body including a cavity in which the chip capacitor resides.

Disclosed herein is an EMI filtered feedthru for an implantable pulse generator. In one embodiment, the EMI filtered feedthru includes a non-filtered feedthru and a modular EMI filter. The non-filtered feedthru may include an electrically conductive housing, an electrically insulating core and a feedthru wire extending through the core. The modular EMI filter assembly may be coupled to the feedthru and include a body and a chip capacitor supported by the body. The capacitor may include a power side in electrical communication with the feedthru wire and a ground side in electrical communication with the housing.

Disclosed herein is a method of manufacturing an EMI filtered feedthru. In one embodiment, the method includes: providing a non-filtered feedthru including an electrically conductive housing, an electrically insulating core and a feedthru wire extending through the core; and coupling a modular EMI filter assembly to the feedthru, wherein the filter assembly includes a body and a chip capacitor supported by the body.

While multiple embodiments are disclosed, still other embodiments of the present invention will become apparent to those skilled in the art from the following Detailed Description, which shows and describes illustrative embodiments. As will be realized, the invention is capable of modifications in various aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1 is an isometric view of an implantable pulse generator employing a feedthru according to the present disclosure.

FIG. 2 is an isometric view of an embodiment of the feedthru of FIG. 1, wherein a filter assembly is shown.

FIG. 3A is a side view of the feedthru of FIG. 2.

FIG. 3B is a side view of an alternative embodiment of the feedthru of FIG. 1.

FIG. 3C is a side view of still another alternative embodiment of the feedthru of FIG. 1.

FIG. 4A is a cross-sectional elevation of the feedthru as taken along section line 4A-4A of FIG. 3A.

FIG. 4B is an enlarged cross-sectional view of the feedthru of FIG. 4A as if viewed in region A of FIG. 4A.

FIG. 4C is a cross-sectional elevation of an alternative embodiment of the feedthru as taken along section line 4C-4C of FIG. 3B.

FIG. 4D is a cross-sectional elevation of an alternative embodiment of the feedthru as taken along section line 4D-4D of FIG. 3C.

FIG. 4E is an enlarged cross-sectional view of the feedthru of FIG. 4C as if viewed in region B of FIG. 4C.

FIG. 4F is an enlarged cross-sectional view of the feedthru of FIG. 4D as if viewed in region C of FIG. 4D.

FIG. 5A is a top isometric view of the filter assembly of the feedthru of FIG. 2.

FIG. 5B is a bottom isometric view of the filter assembly of the feedthru of FIG. 2.

### DETAILED DESCRIPTION

The present disclosure describes a feedthru 55 of an implantable pulse generator 5, such as a pacemaker, a defibrillator or an ICD. The feedthru 55 disclosed herein includes an EMI filter assembly 75. The filter assembly 75 filters unwanted signals, such as EMI signals, that may interfere with the electrical components 17 housed within the can 15 of the implantable pulse generator 5. In one embodiment, the feedthru 55 may also include feedthru wires 60. The feedthru wires 60 electrically connect the components of the header 10 (e.g., the connector blocks 20) with the electrical components 17 (e.g., output flex, hybrid, etc.) housed within the can 15. The feedthru 55 provides an electrically insulated passageway for electrical communication via the wires 60 through the wall of the can 65.

Generally, a discoidal filter assembly is utilized as a component of known filtered feedthrus to filter out EMI signals. The filtered feedthru 55 disclosed herein employs a less expensive, off-the-shelf chip capacitor 90 as an EMI filter element, thereby reducing material and manufacturing costs. The EMI filter assembly 75 disclosed herein may be readily applied to a standard non-filtered feedthru to convert the non-filtered feedthru into a filtered feedthru.

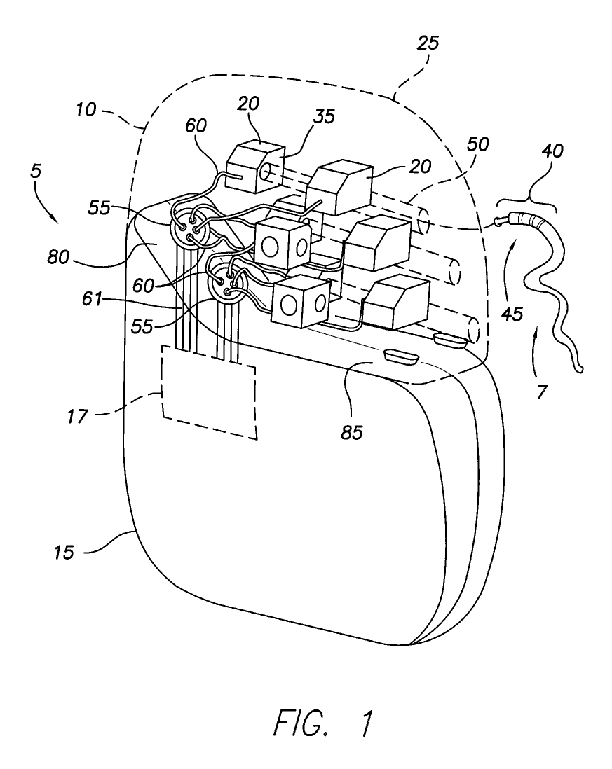

For a general discussion of an implantable pulse generator 5 that utilizes the EMI filtered feedthru 55 disclosed herein, reference is first made to FIG. 1, which is an isometric view of such an implantable pulse generator 5. As indicated in FIG. 1, the pulse generator 5 includes a header 10 and a can or housing 15. The header 10 includes connector blocks 20 and a molded portion 25 (shown in phantom) that encloses the blocks 20. Each block 20 includes an opening 35 configured to receive therein and mate with a connector end 40 of a lead proximal end 45, thereby forming an electrical connection between the connector block 20 and the lead connector end 40 and mechanically securing the proximal end 45 of the lead 7 to the header 10 of the pulse generator 5.

The header molded portion 25 (shown in phantom) may be formed of a polymer material. Passages 50 (shown in phantom) extend from the exterior of the molded portion 25 to the openings 35 in the blocks 20, providing a pathway for the lead distal ends 40 to pass through the molded portion 25 and enter the openings 35.

The can 15 includes feedthrus 55 mounted in the wall of the can 15. Conductors 60 (e.g., round wires, flat ribbon wires, flex cables or etc.) extend from the header sides of the feedthrus 55 to respective connector blocks 20. The can 15 provides a hermetically sealed enclosure for the pulse generator's electronic components 17 (e.g., output flex, hybrid, or various other electronic components) housed within the can 15. Conductors 61 (e.g., round wires, flat ribbon wires, flex cables or etc.) extend from the can sides of the feedthrus 55 to the electronic components 17. Typically, the wall of the can 15 is made of titanium or another biocompatible metal.

As shown in FIG. 1, in one embodiment, the feedthrus 55 are mounted in an inclined portion 80 of the can 15. In other embodiments, the feedthrus 55 may be mounted in a flat portion 85 of the pulse generator 5, or the feedthrus 55 may be mounted in both the inclined and flat portions 80, 85 of the can 15. In other embodiments, the feedthrus 55 may also be mounted in the vertical side walls of the can 15.

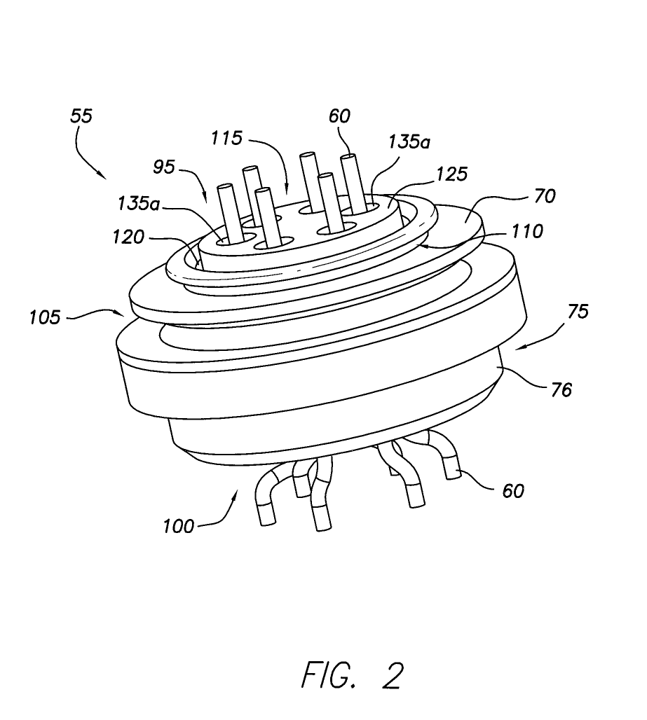

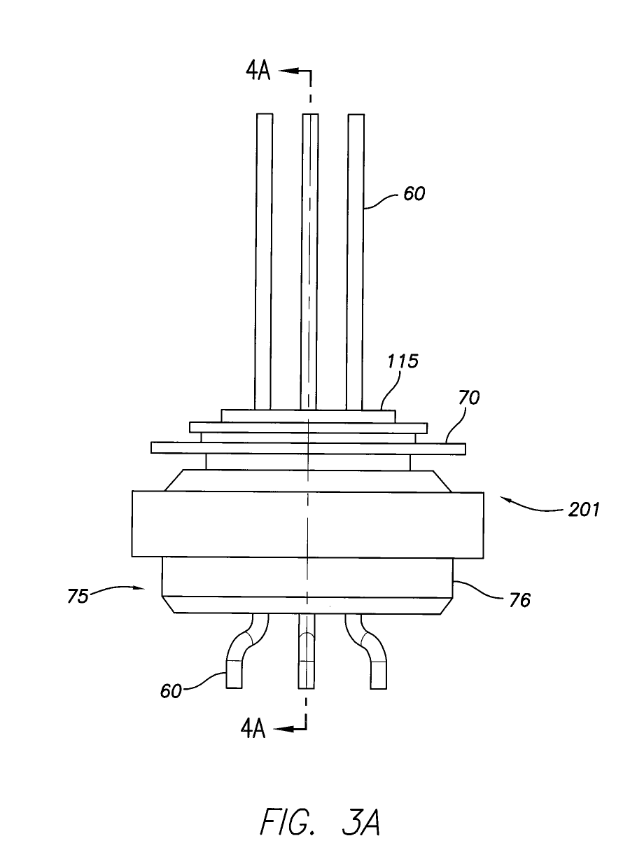

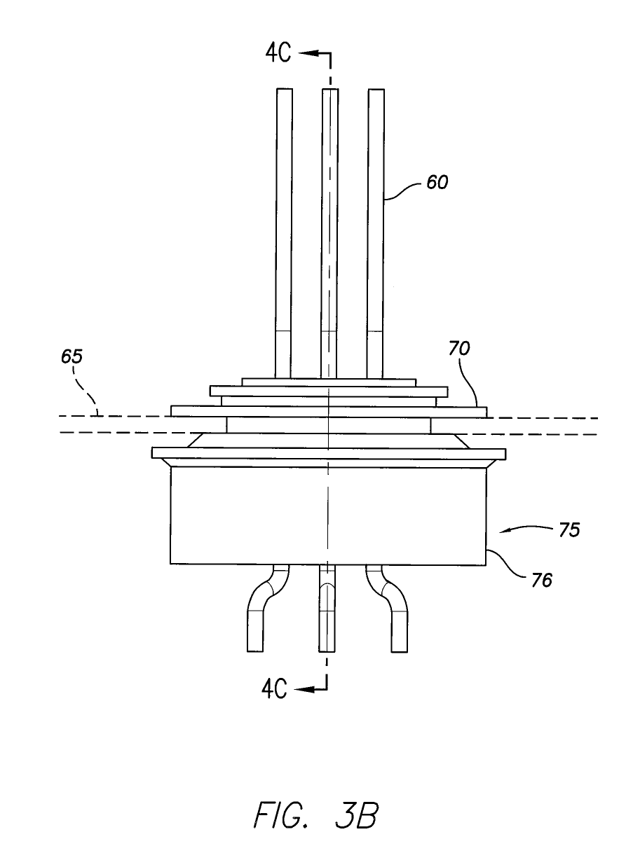

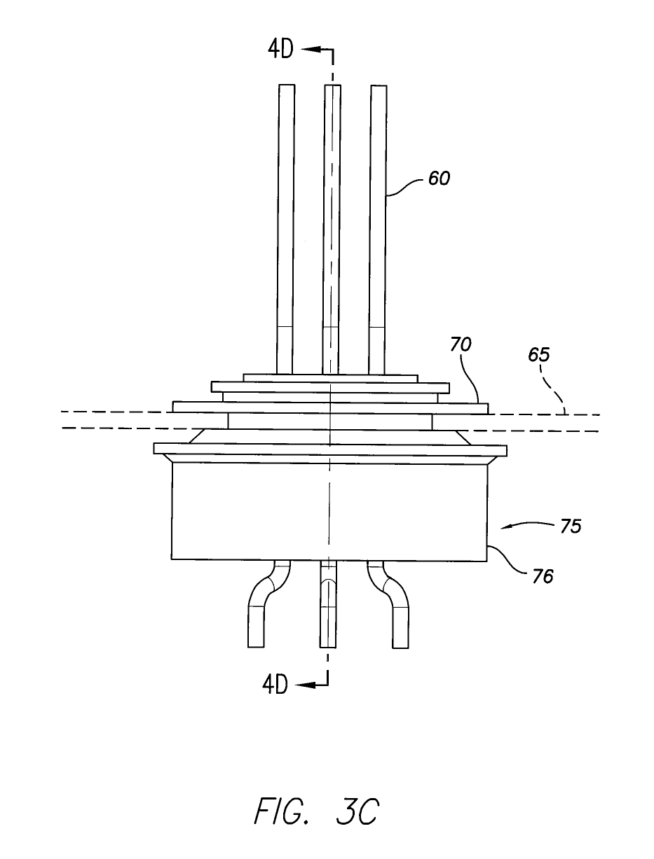

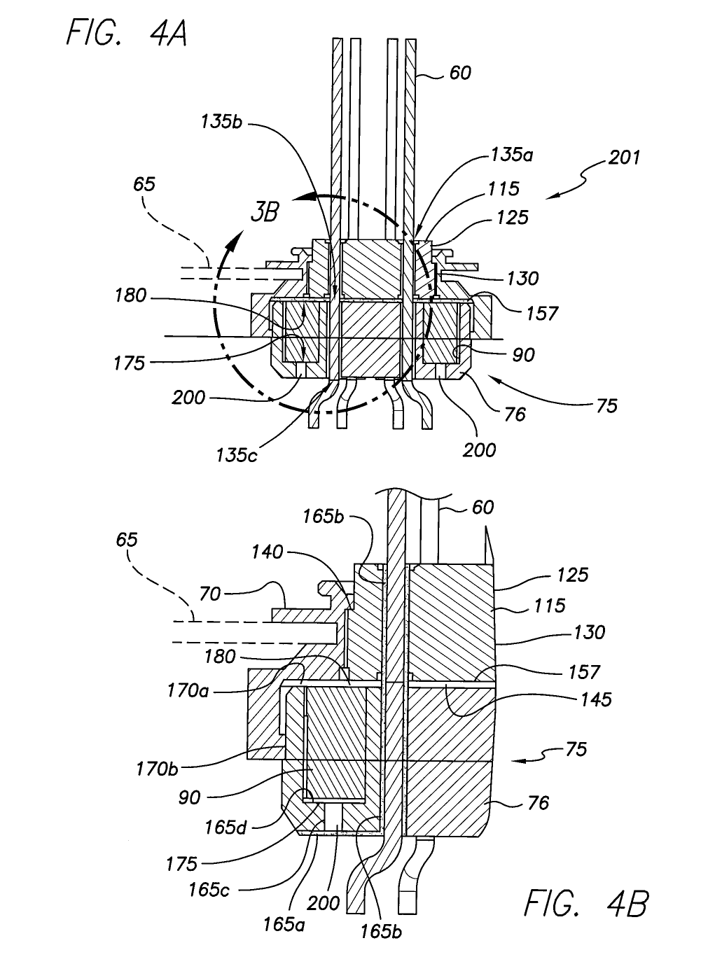

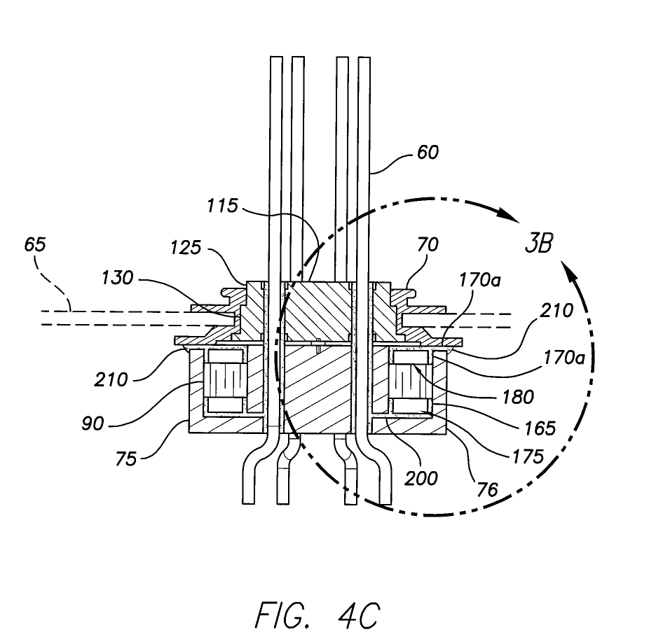

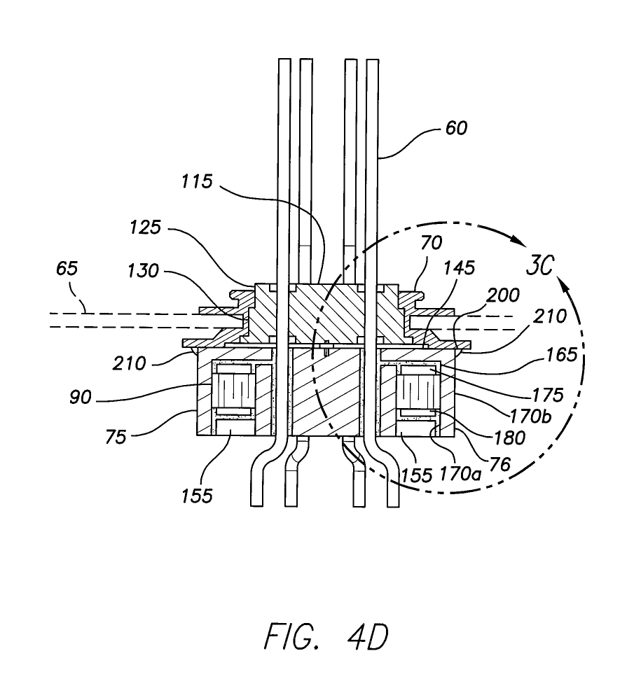

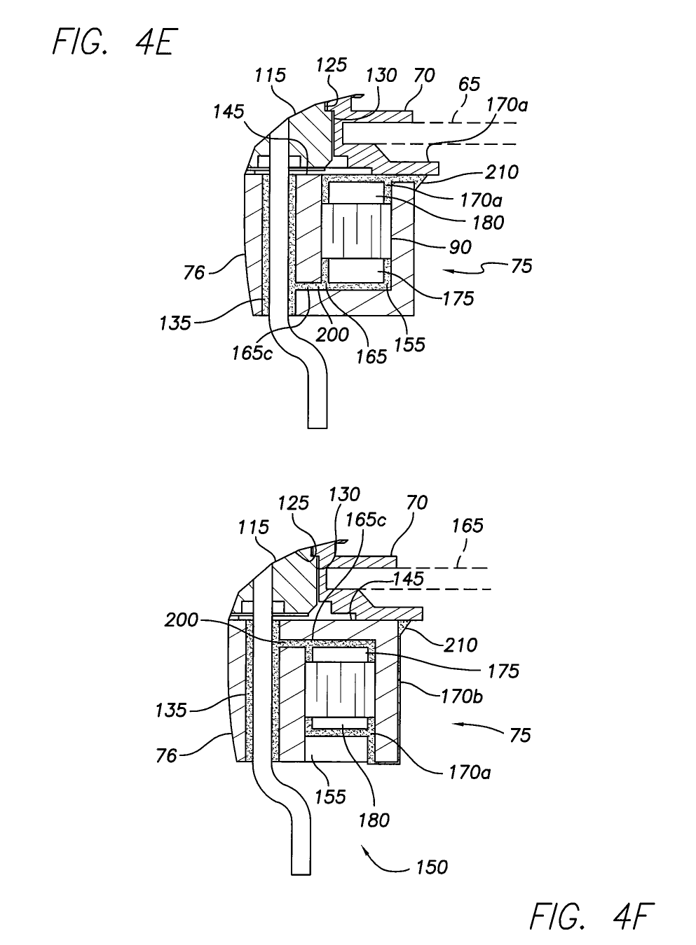

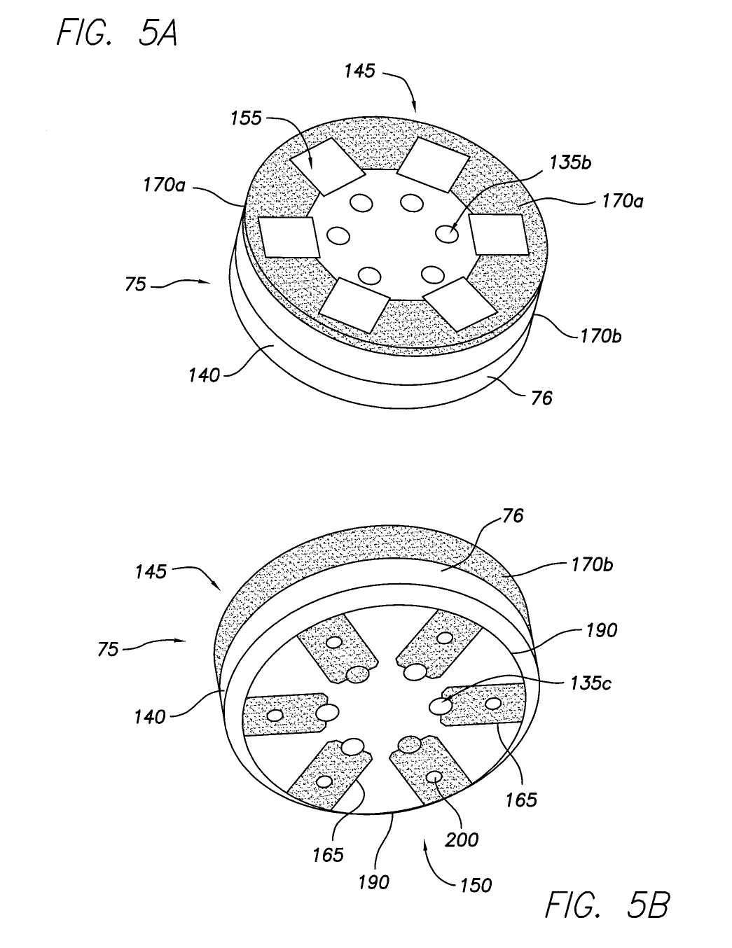

For a detailed discussion of the EMI filtered feedthru 55 as disclosed herein, reference is made to FIGS. 2-5B. FIGS. 2 and 3A are, respectively, an isometric and a side view of the feedthru 55 and FIGS. 3B-3C are side views of respective alternative embodiments of the feedthru 55. FIGS. 4A, 4C and 4D are cross-sectional elevations of various alternative embodiments of the feedthru 55 as taken along section lines 4A-4A, 4C-4C and 4D-4D, respectively, of FIGS. 3A, 3B and 3C, respectively. FIGS. 4B, 4E, and 4F are enlarged cross-sectional views of various alternative embodiments of the feedthru 55 of FIGS. 4A, 4C and 4D, respectively, as if viewed in region A, B and C, respectively, of FIGS. 4A, 4C and 4D, respectively. FIGS. 5A-5B are, respectively, top and bottom isometric views of the filter assembly 75.

As indicated in FIG. 2, in one embodiment, the feedthru 55 includes a header side 95, a can side 100, and a circular side 105 that may vary in diameter such that it appears as a plurality of stacked rings with different diameters. A groove or slot 110 may be defined by the varying diameter. As depicted in FIG. 4A, when the feedthru 55 is assembled in the can 15, the groove or slot 110 receives the wall 65 of the can 15. Feedthru wires 60 extend from the header side 95 and the can side 100.

As can be understood from FIGS. 2-5B, in one embodiment, the feedthru 55 includes feedthru wires 60, a feedthru housing 70, a core 115, an EMI filter assembly 75 and ground and power circuits. As best understood from FIGS. 4A-4F, in some embodiments, the feedthru housing 70, core 115 and feedthru wires 60 may be considered in combination as a non-filtered feedthru 201, and the EMI filter assembly 75, which may be considered to have a modular characteristic or configuration that includes the body 76 and the capacitor chips 90 held therein, may be added to the non-filtered feedthru 201 to form an EMI filtered feedthru 55.

As indicated in FIGS. 2-4F, the housing 70 includes the circular side 105, the groove or slot 110, and a central or core receiving opening 120. The housing 70 may be molded, machined, or otherwise formed and may be unfiltered. The housing may be titanium, a titanium alloy, stainless steel, or MP35N.

The outer circumference of the housing 70 is defined by the groove or slot 110 and the circular side 105. The central opening 120 of the housing 70 extends axially through the housing 70 and may have a stepped construction. The central opening 120 defines an aperture which is occupied by the core 115.

In one embodiment, the feedthru 55 includes feedthru wires 60. The feedthru wires 60 may be Pt/Ir wires, such as 90% Pt/10% Ir wires. The electrical components 17 in the can 15 and the blocks 20 in the header 10 may be coupled to the wires 60 by soldering, brazing, welding or other suitable methods.

As can be understood from FIGS. 2 and 4A-4F, the core 115 includes a first cylindrical portion 125, a second cylindrical portion 130 and feedthru wire openings 135 extending longitudinally therethrough. The feedthru wires 60 extend through the opening 135, which provides an insulated passageway for the wires 60 through the core 115 and, as a result, the feedthru 55. The core 115 may be ceramic, sapphire, or glass.

The outer circumference of the core 115 may be cylindrically stepped such that it includes a first cylindrical portion 125 and a second cylindrical portion 130. The first cylindrical portion 125 has a smaller diameter than the diameter of the second cylindrical portion 130.

The core 115 is received in the central opening 120 of the housing 70 such that the first cylindrical portion 125 is exposed at the header side 95 of the feedthru 55 and the second cylindrical portion 130 abuts a step 140 in the central opening 120 of the housing 70.

As shown in FIGS. 2 and 4A, the core 115 includes feedthru wire openings 135a that receive the feedthru wires 60. The feedthru wire openings 135a generally correspond to the feedthru wire openings 135b, 135c in the filter assembly 75 to form continuous feedthru wire openings 135 that extend through the feedthru 55.

As can be understood from FIGS. 5A and 5B, the feedthru 55 includes a ground circuit and a power circuit. The ground circuit includes the feedthru housing 70 and the ground traces 170, which extend along portions of the circumferential side 140 and core side 145 of the filter assembly 75. As indicated in FIG. 4B, the ground traces 170 electrically couple the ground ends 180 of the chip capacitors 90 to the feedthru housing 70, which is electrically coupled to the can wall 65.

As can be understood from FIGS. 5A and 5B, the power circuit includes the feedthru wires 60 and the power traces 165, which extend along portions of the electronic interface side 150 of the filter assembly 75 and into holes through which the feedthru wires 60 extend and holes 200 leading to the capacitor chip pockets 155. As indicated in FIG. 4B, the power traces 165 electrically couple the power ends 175 of the chip capacitors 90 to the feedthru wires 60 extending through the feedthru 55. A detailed discussion regarding each of the components of the power and ground circuits is provided below.

For a detailed discussion of the filter assembly 75, reference is now made to FIGS. 4A-5B. The filter assembly 75 includes a body 76, cavities 155, chip capacitors 90, feedthru wire openings 135, an outer circumferential side 140, a core side 145 and an electronic interface side 150. The outer circumferential side 140 of the filter assembly 75 and the outer circumference of the housing 70 define the outer circumference of the feedthru 55.

As can be understood from FIGS. 2-5B, in one embodiment, the body 76 of the filter assembly 75 is a disc. In alternative embodiments, the body 76 of the filter assembly 75 may be a shape other than a disc, such as a hexagon or a rectangle. The filter assembly body 76 may be formed of any electrically insulating material, such as ceramic, sapphire or glass that is brazable. The filter assembly body 76 may be machined, molded or otherwise formed to fit the space and design constraints of the implantable pulse generator 5.

As can be understood from FIGS. 4A-4B, the filter assembly body 76 may be at least partially recessed within the housing 70. In other embodiments, as shown in FIGS. 4C-4F, the filter assembly body 76 may be mounted flush to the housing 70.

As shown in FIGS. 4A, 4B, 4E and 5A, cavities 155 are defined in the body 76 of the filter assembly 75 and open outwardly on the core side 145 of the body 76 to receive therein chip capacitors 90. In some embodiments, as shown in FIG. 4F, cavities 155 may open outwardly on the electronic interface side 150 of the body 76 to receive therein chip capacitors 90. The cavities 155 may be shaped to matingly receive the capacitors 90. The cavities 155 have a bottom surface opposite their openings in the core side 145 or opposite their openings in the electronic interface side 150, as appropriate. As shown in FIGS. 4A, 4B and 5A, in one embodiment, holes 200 extend through the material of the body 76 from the bottom surface of the cavities 155 to form an opening in the electronic interface side 150 of the body 76. As shown in FIGS. 4C-4F, in some embodiments, holes 200 extend through the material of the body 76 from the top or bottom surface, respectively, of the cavities 155 to form an opening in the feedthru wire openings 135.

As can be understood from FIGS. 2-5B, the feedthru wire openings 135 extend through the core 115 and the filter assembly body 76, providing a passageway for the feedthru wires 60 to extend through and electrically connect the components 20,17 of the header 10 and the can 15, respectively. The surfaces of the feedthru wire openings 135 may be coated with an electrically conductive material, such as nickel, gold, platinum, etc.

The feedthru wire openings 135 are arranged radially about a center point. In alternative embodiments, the openings 135 may be arranged in a different pattern, e.g. not radially, or the openings 135 may be located on an outside rim or edge. In one embodiment, there are six openings 135. In alternative embodiments, there may be fewer than six openings 135 or there may be more than six openings 135.

As can be understood from FIGS. 4A-5B, the chip capacitors 90 include a power end 175 and a ground end 180. The power end 175 of the chip capacitor 90 is electrically connected to the power trace 165. The ground end 180 of the chip capacitor 90 is electrically connected to the ground trace 170. In one embodiment, the minimum distance between opposite electrical potentials is approximately 0.03 inches.

In one embodiment, the chip capacitors 90 are easy to obtain, that is, they are “off-the-shelf” or commercially available chip capacitors. For example, chip capacitors 90 such as model 0805, manufactured by Novacap of Valencia, Calif., USA, may be utilized in the feedthru 55. The chip capacitors 90 serve as an EMI filter element. EMI is a (usually undesirable) disturbance caused in a radio receiver or other electrical circuit by electromagnetic radiation emitted from an external source. An EMI signal may interfere with the electrical components in the can of the implantable pulse generator. Thus, an EMI filter element, such as a chip capacitor, may reduce or eliminate the interference caused by such a signal. Also, an “off-the-shelf” chip capacitor may be less expensive and easier to acquire than a discoidal filter assembly, thus reducing the material and manufacturing costs of the feedthru 55.

As illustrated in FIG. 5A, the core side 145 of the filter assembly body 76 includes chip capacitor openings 155, feedthru wire openings 135b and ground traces 170. The chip capacitor openings 155 receive the chip capacitors 90. The chip capacitors 90 may be coupled to the openings 155 by an electrically conductive epoxy or solder. In one embodiment, there may be six, square chip capacitor openings 155. In alternative embodiments, the chip capacitor openings 155 may be a shape other than a square, such as a rectangle, or any other shape as needed to accommodate the size and shape of a chip capacitor 90. In other alternative embodiments, there may be more than six chip capacitor openings or there may be less than six chip capacitor openings. The surfaces of the chip capacitor openings 155 may be coated with an electrically conductive material, such as nickel, gold, platinum, etc.

In some embodiments, as shown in FIGS. 4C-4F, conductive epoxy or solder 210 electrically connects the filter assembly 75 and the housing 70. The conductive epoxy or solder 210 may form a portion of the ground trace 170.

As can be understood from FIGS. 4B, 4D, 4F and 5A, the ground trace 170 extends over the outer circumferential surface 140 of the body 76. The ground trace 170b extending over the filter assembly outer circumferential surface 140 is in electrical contact with, and welded or brazed to, the housing 70. The housing 70 is in electrical contact with the can wall 65, which serves as the ground for the implantable pulse generator 5.

As shown in FIGS. 4D and 4F, in some embodiments, the ground trace 170a extends partially about the outside corners of the chip capacitor openings 155. In some embodiments, as shown in FIGS. 4C and 4E, the ground trace 170 a further extends across a portion of the core side 145 of the body 76 of the filter assembly 75. In other embodiments, as shown in FIG. 5A, the ground trace 170 a extends partially about the chip capacitor openings 155 such that the trace 170 a forms a ring about the chip capacitor openings 155. The ground trace 170 in any of its locations serves as a part of the ground circuit by coupling the ground side 180 of the chip capacitors 90 to the can wall 65, via the feedthru housing 70, which is also a part of the ground circuit. The ground trace 170 in any location may be formed by any method, such as photoetching, deposition, etc., and the ground trace 170 may be made of gold, nickel, or platinum.

As shown in FIGS. 4B and 5B, the electronic component interface side 150 of the filter assembly body 76 includes power traces 165, feedthru wire openings 135c, and holes 200 leading to the chip cavities 155. As can be understood with reference to FIG. 1, the electronic component interface side 150 of the filter assembly 75 is electrically coupled to the electrical components 17 (e.g., a printed circuit board) in the can 15 of the implantable pulse generator 5 via the feedthru wires 60.

As illustrated in FIGS. 4B and 5B, power traces 165a extend across the face of the electronic component interface side 150 from approximately the feedthru wire opening 135c to the outer circumference 190 of the interface side 150 of the filter assembly 75. The power trace 165a electrically couples the feedthru wire 60 to the power side 175 of the chip capacitor 90. The power trace 165a may extend along the face of the interface side 150 in the form of a rectangle.

As shown in FIG. 4B, power traces 165b, in the form of electrically conductive coatings, may extend along the surfaces of the feedthru wire openings 135 to join with the power traces 165a on the interface side 150 of the filter assembly body 76. Similar power traces 165c may also extend along the holes 200 from the rectangular power traces 165a on the interface side 150 to power traces 165d on the bottom of the chip cavities 155.

As can be understood from FIGS. 4C-4F, in some embodiments, power traces 165e may extend along the holes 200 from the feedthru wire openings 135 to the power side of the chip capacitor 90, thereby electrically coupling the feedthru wire 60 to the power side of the chip capacitor 90.

The power traces 165a, 165b, 165c, 165d, or 165e form a power side electrical circuit along with the feedthru wires 60 that electrically couples the power side 175 of the chip capacitor 90 with the connector blocks 20 in the header 10 and the electrical components 17 in the can 15 via the feedthru wires 60. The power traces 165 may be formed of any conductive material, such as gold, nickel, platinum, etc. which is capable of being formed into a trace by any method, such as photoetching, deposition, plating, etc.

As can be understood from FIGS. 4A and 4B, an electrical insulation polymer 157 is located between the core side 145 of the filter assembly 75 and the second cylindrical portion 130 of the core 115 when the feedthru 55 is assembled. As shown in FIGS. 4C-4F, in alternative embodiments, a non-conductive epoxy may be located between the second cylindrical portion 130 of the core 115 and the core side 145 of the assembly 75.

As can be understood from FIGS. 2 and 3A-3C, to assemble the feedthru 55, the housing 70 and the core 125 are coupled by brazing, soldering, welding or other appropriate method, thereby creating a hermetic seal. The filter assembly 75 may be coupled directly or indirectly (e.g. via an electrical insulation polymer 157) to the core 115. The filter assembly 75 may be electrically coupled to the housing 70 by brazing, soldering, welding or other suitable method. The chip capacitors 90 may be assembled into the chip capacitor openings 155 by soldering or an electrically conductive epoxy. In one embodiment, the feedthru wires 60 may be coupled to the core 125 by soldering, welding, brazing, conductive epoxy or other suitable method.

As can be understood from FIGS. 2 and 3A-3C, and with reference to FIG. 1, the feedthru 55 is mounted into the can wall 65 and feedthru wires 60 electrically connect the connector blocks 20 in the header 10 to the electronic components 17 in the can 15. The components 20,17 electrically communicate with each other and the power end 175 of the chip capacitor 90 through the power circuit that is formed by the feedthru wires 60 and the power trace 165. The can wall 65 is electrically connected to the housing 70 and electrically communicates with the ground end 180 of the chip capacitor 90 through the ground circuit formed by the feedthru housing 70 and the ground trace 170.

Although the present invention has been described with reference to preferred embodiments, persons skilled in the art will recognize that changes may be made in form and detail without departing from the spirit and scope of the invention.

What is claimed is:

1. An EMI filtered feedthru for an implantable pulse generator, the EMI filtered feedthru comprising: a filter assembly including a chip capacitor and a body including a cavity in which the chip capacitor resides.

2. The EMI filtered feedthru of claim 1, further comprising an electrically insulating core, an electrically conductive housing bordering the core, and a feedthru wire extending through the core, wherein the filter assembly is coupled to at least one of the housing and core.

3. The EMI filtered feedthru of claim 2, wherein the filter assembly further includes a power trace and ground trace, wherein the power trace is in electrical communication with the feedthru wire and a power side of the chip capacitor and the ground trace is in electrical communication with a ground side of the chip capacitor and the housing.

4. The EMI filtered feedthru of claim 2, wherein the feedthru wire extends through the filter assembly.

5. The EMI filtered feedthru of claim 2, wherein the chip capacitor is enclosed in the cavity of the body by at least one of the core and housing.

6. The EMI filtered feedthru of claim 1, wherein the filter assembly further includes a power trace and a ground trace, and wherein the power trace is in electrical communication with a power side of the chip capacitor and a ground trace is in electrical communication with a ground side of the chip capacitor.

7. The EMI filtered feedthru of claim 6, wherein the body is formed of an electrically insulating material.

8. The EMI filtered feedthru of claim 1, wherein the chip capacitor is an off-the-shelf type chip capacitor.

9. The EMI filtered feedthru of claim 1, wherein the cavity is a plurality of cavities and the chip capacitor is a plurality of chip capacitors residing in the plurality of cavities.

10. The EMI filtered feedthru of claim 9, wherein the plurality of cavities are generally equally radially dispersed about a center of the body.

11. The EMI filtered feedthru of claim 6, wherein a first end of the cavity defines an opening in a first face of the body and a second end of the cavity opposite the first end defines a recessed surface of the body.

12. The EMI filtered feedthru of claim 11, wherein a portion of the ground trace boarders the opening.

13. The EMI filtered feedthru of claim 11, wherein a portion of the power trace is on the recessed surface.

14. The EMI filtered feedthru of claim 11, wherein the body includes a hole extending from the recessed surface to a second face of the body.

15. The EMI filtered feedthru of claim 14, wherein a portion of the power trace extends along a surface of the hole.

16. An implantable pulse generator comprising: an EMI filtered feedthru including an EMI filter assembly having a chip capacitor and a body including a cavity in which the chip capacitor resides.

17. The implantable pulse generator of claim 16, wherein the EMI filtered feedthru further includes an electrically insulating core, an electrically conductive housing bordering the core, and a feedthru wire extending through the core, and wherein the filter assembly is coupled to at least one of the housing and core.

18. The implantable pulse generator of claim 17, wherein the EMI filter assembly further includes a power trace and ground trace, and wherein the power trace is in electrical communication with the feedthru wire and a power side of the chip capacitor and the ground trace in is electrical communication with a ground side of the chip capacitor and the housing.

19. The implantable pulse generator of claim 16, wherein the EMI filter assembly further includes a power trace and a ground trace, and wherein the power trace is in electrical communication with a power side of the chip capacitor and a ground trace is in electrical communication with a ground side of the chip capacitor.

20. An EMI filtered feedthru for an implantable pulse generator, the EMI filtered feedthru comprising:
a non-filtered feedthru including an electrically conductive housing, an electrically insulating core and a feedthru wire extending through the core; and
a modular EMI filter assembly coupled to the feedthru and including a body and a chip capacitor supported by the body, the capacitor including a power side in electrical communication with the feedthru wire and a ground side in electrical communication with the housing.

21. The EMI filtered feedthru of claim 20, wherein body includes a cavity in which the chip capacitor is located.

22. A method of manufacturing an EMI filtered feedthru, the method comprising:
providing a non-filtered feedthru including an electrically conductive housing, an electrically insulating core and a feedthru wire extending through the core; and
coupling a modular EMI filter assembly to the feedthru, wherein the filter assembly includes a body and a chip capacitor supported by the body.

23. The method of claim 22, wherein the body includes a cavity in which the chip capacitor is located.
